(19) United States
(12) Patent Application Publication
(10) Pub. No.: US 2020/0206267 A1
(43) Pub. Date: Jul. 2, 2020

(54) ALLOGENEIC CAR-T PLATFORM USING HLA-MATCHED BANK OF IPSCS, AND RELATED COMPOSITIONS, SYSTEMS, AND METHODS

(71) Applicant: Orig3n, Inc., Boston, MA (US)

(72) Inventors: Robin Y. Smith, Boston, MA (US); Marcie A. Glicksman, Boston, MA (US)

(21) Appl. No.: 16/643,814

(22) PCT Filed: Mar. 14, 2018
(86) PCT No.: PCT/US18/22319

§ 371 Date: Mar. 2, 2020

Related U.S. Application Data
(60) Provisional application No. 62/553,550, filed on Sep. 1, 2017.

(51) Int. Cl. A61K 35/17; C07K 14/725; G06F 17/18

(52) U.S. Cl. CPC A61K 35/17; G06F 2219/00; G06F 17/18; C07K 14/7051

(57) ABSTRACT

Presented herein are systems and methods of producing “universal” and/or “off-the-shelf” CAR-T compositions suitable for cancer therapy to be administered to one or more individuals. A CAR-T composition is a composition comprising one or more types of chimeric antigen receptor T cells (CAR-T). The iPSCs and/or cell lines, and any iPSC-derived CAR-T compositions derived therefrom, are identified as compatible with one or more individuals using an identification of a cell type indicative of compatibility (e.g., HLA match and/or ABO blood match and/or RHD blood match). The compatible cells are then retrieved from a managed HLA-indexed (and/or otherwise indexed) repository or are derived from a biological sample of a donor. The retrieved compatible cells are then used to derive iPSC-derived CAR-T compositions, wherein the derived compositions are suitable for therapy of one or more individuals.

### CROSS REFERENCE TO RELATED APPLICATIONS

This application claims the benefit of U.S. Provisional Application No. 62/553,550 filed Sep. 1, 2017, the contents of which are hereby incorporated by reference herein in their entirety.

### FIELD OF INVENTION

The invention relates generally to apparatus, systems, and methods directed to allogeneic CAR-T using an HLA-indexed bank of induced pluripotent stem cells (iPSCs).

### BACKGROUND

Chimeric antigen receptors T cell (CAR-Ts) can be generated from a donor (e.g., a patient) and used in cancer (e.g., blood cancer) treatment. CAR-T cell therapy production involves a number of steps. First, T cells are collected from a donor (e.g., a patient), and then sent to a laboratory where they are engineered to produce chimeric antigen receptors (CARs) on the surface of the T cells. The CAR-T cells are then infused either back into the same donor (e.g., patient) or into multiple patients for recognition and killing of cancerous cells that express the targeted antigen, while continuing to multiply within the body of the patient. Currently, CARs armed with the protein CD19 have been effective in recent clinical trials.

There are two strategies for CAR-T cell therapy: autologous and allogeneic strategies.

An “autologous” strategy involves engineering CAR-T cells using a patient's own T cells. This strategy has to be performed for each patient and therefore is expensive and time consuming. Some patients have low lymphocyte numbers or poor lymphocyte quality and therefore autologous therapies are not an option.

An “allogeneic” strategy involves using T cells from a single (healthy) donor that are infused into one or more patients. Allogeneic cells can be prepared and stored for large groups of individuals and can be made in advance. Moreover, fewer allogeneic lines are needed to serve a population. As a result, the allogeneic strategy reduces the expensive and time consuming manufacturing required by the autologous strategy, and provides cell transfer across histocompatibility barriers.

A challenge in producing allogeneic CAR-T cell therapies is that the endogenous T cell receptor (TCR) on allogeneic T cells may recognize alloantigens of the recipient, leading to graft-versus-host disease (GVHD). As a result, expression of HLA on the surface of allogeneic T cells may cause rapid rejection by the host immune system. Thus, in order to reduce the likelihood of rejection of allogeneic CAR-T cell therapy, it is important the patient receives histocompatible (or “HLA-matched”) cells, and “sees” the allogeneic CAR-T cells as “self”.

The term “super donors” refers to human leukocyte antigen (HLA) types (or cell lines or individuals having those HLA types) that do not trigger strong rejection reactions. Super donors have a HLA haplotype that is common among the population and will match a sizable portion of a particular population. This is analogous to banking a blood transfusion from a donor who has blood type O-negative, which can be tolerated by patients of all blood types.

Humans are almost always heterozygous for a particular HLA gene—that is, humans usually express two different alleles. For a successful match, eight (8) HLA alleles are best for matching (4 alleles on each of the donor and recipients chromosomes). With homozygous donors, only 4 alleles are required to be matched, therefore increasing the number of recipients that would be a match to the donor. Individuals that are homozygous for all three key HLA alleles that govern rejection means that only three genes need to be matched instead of six genes. Accordingly, iPSC lines derived from these so-called “super donors” can be used to reduce immunogenicity. It is believed that about 200 such iPSC lines could cover a high percentage (e.g., at least 90%, at least 95%, or more) of the U.S. and/or European population, and about 90 to 100 such iPSC lines could cover a high percentage (e.g., at least 90%, at least 95%, or more) of the Japanese population.

Donor registries are services that seek to match registered donors with patients in need of an allogeneic transplant. Matching based on human leukocyte antigen (HLA) typing is typically performed to find suitable donors. Because there are many different HLA types, it is often difficult to find suitable matches, particularly when no family members of the patient are an HLA-identical match. This is one of many reasons the allogeneic CAR-T strategy has yet to become a reliable cancer treatment.

In attempts to solve these challenges, methods of engineering HLA-matched T cells have emerged. A common method is to use viral vectors such as retrovirus, lentivirus or transposons that become part of the host cell genome. In some applications, non-integrating vectors are preferred because they are less likely to disrupt the host's genome. Alternatively, either plasmids or mRNA, which will be diluted and lost with multiple cell divisions, can be used. A third possibility is to use a vector that is stably maintained in the T cell. Alternatively, some methods using CRISPR/Cas9 have emerged for multiplexed gene editing (e.g., editing of TCR, e.g., editing of B2M, e.g., editing PM) to generate CAR-T cells deficient in the expression of endogenous TCR and HLA class I in hopes of producing a universal CAR-T cell therapy that can transfer across histocompatibility barriers (see Ren et al. “Multiplex Genome Editing to Generate Universal CAR T Cells Resistant to PD1 Inhibition”, Clinical Cancer Research, published online Nov. 4, 2016, the contents of which are hereby incorporated by reference in its entirety).

Therefore, there is a need for systems and methods of sourcing compatible cells from donors using a matched HLA bank. Such a platform would provide “universal” and “off-the-shelf” CAR-T cells that would be available to patients when needed.

### SUMMARY

Presented herein are systems and methods of producing “universal” and/or “off-the-shelf” chimeric antigen receptor T cell (CAR-T) compositions suitable for cancer (e.g., blood cancer) therapy to be administered to a specific individual and/or specific group of individuals. A CAR-T composition is a composition comprising one or more types of chimeric antigen receptor T cells (CAR-T). The iPS cells and/or cell lines, and any iPSC-derived CAR-T compositions derived therefrom, are identified as compatible with a specific individual or specific group of individuals using an identification of a cell type indicative of compatibility such as an HLA match and/or ABO blood match and/or RHD blood group match. The compatible iPS cells or cell lines are then retrieved from a managed HLA-indexed (and/or otherwise indexed) repository or are derived from a biological sample of a suitable donor. The retrieved compatible cells are then used to derive the “universal” and/or “off-the-shelf” iPSC-derived CAR-T composition, wherein the “universal” and/or “off-the-shelf” iPSC-derived CAR-T compositions are suitable for therapy of a specific individual and/or specific group of individuals.

The techniques described herein allow for the tuning of CAR-T compositions to a specific individual or a specific group of individuals, thus enabling improved methods of CAR-T based therapy, e.g. due to an enhanced compatibility of the specific individual or group of individuals with the cells from which the desired CAR-T composition is derived. Also, allogeneic iPS cells and/or cell lines that are compatible with a large portion of a specific population, e.g. super donors, can be prepared and stored in advance for large groups of individuals. These super donor-derived CAR-T compositions can then be made immediately available to people who need them, thus reducing production times of the iPSC-derived CAR-T compositions.

In one aspect, the invention is directed to a method of manufacturing an induced pluripotent stem cell (iPSC)-derived chimeric antigen receptor T cell (CAR-T) composition tailored for treatment of a particular subject or particular group of subjects, said method comprising the steps of: (a) identifying, as compatible with the particular subject or particular group of subjects, one or more iPSCs and/or one or more iPSC-derived cells (e.g., one or more induced pluripotent stem cells (iPSCs) [the term “cells” being inclusive of both cells and cell lines]); (b) retrieving compatible cells corresponding to the one or more iPSCs and/or one or more iPSC-derived cells identified as compatible with the particular subject or particular group of subjects; and (c) producing the iPSC-derived CAR-T composition using the retrieved compatible cells.

In certain embodiments, the compatible cells comprise one or more members selected from the group consisting of iPSCs, stem cells, blood progenitors, iPSCs, hematopoietic stem cells (HSCs), mesenchymal stem cells (MSCs), Retinal Pigment Epithelium (RPE), and embryoid bodies.

In certain embodiments, step (b) comprises deriving the compatible cells from a biological sample of a donor. In certain embodiments, the donor is not the particular subject or a member of the particular group of subjects.

In certain embodiments, step (b) comprises obtaining the compatible cells from a physical repository.

In certain embodiments, step (b) comprises retrieving, by a processor of a computing device, one or more data entries corresponding to the compatible cells using a processor-based query from a user, wherein the query comprises an identification of a cell type indicative of compatibility with the subject.

In certain embodiments, the identification of cell type indicative of compatibility with the particular subject or particular group of subjects comprises one or more of (i) to (iii): (i) an HLA match, (ii) an ABO blood type match, and (iii) an RHD blood group match.

In certain embodiments, the iPSC-derived CAR-T composition comprises the retrieved compatible cells.

In certain embodiments, the method comprises producing T cells from the one or more iPSCs, and/or one or more iPSC-derived cells identified as compatible with the particular subject or particular group of subjects.

In certain embodiments, the compatible cells are engineered using CRISPR/Cas9 technology. In certain embodiments, the method comprises producing the iPSC-derived CAR-T composition using the engineered compatible cells.

In another aspect, the invention is directed to a method of manufacturing an induced pluripotent stem cell (iPSC)-derived chimeric antigen receptor T cell (CAR-T) composition tailored for treatment of a particular subject or particular group of subjects, said method comprising the steps of: (a) storing, by a processor of a computing device, a database comprising a data entry corresponding to each of a plurality of characterized cells in a physical repository, wherein the characterized cells comprise iPSCs and/or iPSC-derived cells; (b) receiving, by the processor, a query from a user comprising an identification of a cell type of the particular subject or particular group of subjects; (c) matching, by the processor, the query to one or more data entries of the database, each of the matching data entries corresponding to each of the plurality of characterized cells having a cell type compatible with the particular subject or particular group of subjects, thereby identifying as compatible with the subject the one or more characterized cells; (d) retrieving, from a physical repository, compatible cells corresponding to the one or more characterized cells identified as compatible with the particular subject or particular group of subjects; and (e) producing the iPSC-derived CAR-T composition using the retrieved compatible cells.

In certain embodiments, the data entry corresponding to each of the plurality of characterized cells comprises a set of characterized HLA loci corresponding to the cell, the query comprises a set of queried HLA loci for the particular subject or the particular group of subjects, and the one or more matched data entries of the database are each representative of one or more characterized compatible cells matching the queried HLA loci.

In certain embodiments, the plurality of characterized cells in the physical repository are immortalized.

In certain embodiments, the set of characterized HLA loci corresponding to each of the plurality of characterized cells comprises a set of at least 3 HLA loci, wherein the at least 3 HLA loci are HLA-A, HLA-B, and HLA-DRB.

In certain embodiments, the set of characterized HLA loci corresponding to each of the plurality of characterized cells comprises a set of at least 9 given loci, wherein the at least 9 given loci are HLA-A, HLA-B, HLA-C, HLA-DRB1, HLA-DRB3, HLA-DRB4, HLA-DRB5, HLA-DQB1, HLA-DPB1.

In certain embodiments, the set of characterized HLA loci corresponding to each of the plurality of characterized cells comprises at least 3 given loci selected from the group consisting of HLA-A, HLA-B, HLA-C, HLA-DRB1, HLA-DRB3, HLA-DRB4, HLA-DRB5, HLA-DQB1, and HLA-DPB1.

In certain embodiments, each of the one or more matching data entries of the database exactly match or partially match the set of queried HLA loci for the particular subject or the particular group of subjects.

In certain embodiments, the method comprises determining a corresponding bar code or other identifier for the one or more characterized compatible cells corresponding to each of the retrieved matching data entries, thereby allowing retrieval of desired cells from the physical repository and/or retrieval of identifying information corresponding to the one or more characterized compatible cells matching the queried HLA loci.

In certain embodiments, the data entry for each of the plurality of characterized cells further comprises ABO blood type and the query further comprises ABO blood type, and wherein the one or more matching data entries of the database representative of the one or more characterized compatible cells match the queried HLA loci and the queried ABO blood type.

In certain embodiments, the data entry for each of the plurality of characterized cells further comprises RHD blood group and the query further comprises RHD blood group, and wherein the one or more matching data entries of the database representative of the one or more characterized compatible cells match the queried RHD blood group and the queried HLA loci.

In certain embodiments, the queried HLA loci correspond to the particular subject or particular group of subjects in need of an HLA matched iPSC-derived CAR-T composition [e.g., the HLA match corresponding to one or more samples represented in the database (e.g., each sample comprising cells, e.g., iPS cells and/or embryoid bodies and/or blood progenitors and/or HSCs) exactly matching, partially matching, identified as compatible with (e.g., compatible HLA type), etc., the HLA loci of the subject].

In certain embodiments, one or more of the queried HLA loci is determined by processing and analyzing a biological sample from the particular subject in need of the HLA match.

In certain embodiments, the queried ABO blood type, is determined by processing and analyzing a biological sample from the particular subject in need of an ABO match.

In certain embodiments, the queried RHD blood group is determined by processing and analyzing a biological sample from the particular subject in need of a RHD blood group match.

In certain embodiments, the physical repository comprises one or more liquid nitrogen storage tanks (e.g., and/or another freezer system).

In certain embodiments, the method comprises producing T cells from each of the one or more characterized compatible cells corresponding to the one or more data entries matching the queried HLA loci.

In certain embodiments, the method further comprises administering the iPSC-derived CAR-T composition to the particular subject or particular group of subjects.

In certain embodiments, the administering step comprises administering the iPSC-derived CAR-T composition to the particular subject or particular group of subjects for treatment of a known disease or condition in the in the particular subject or particular group of subjects . In certain embodiments, the known disease or condition is a cancer. In certain embodiments, the known disease or condition is a cancer comprising a member selected from the group consisting of acute myeloid leukemia, acute lymphoblastic leukemia, chronic myeloid leukemia, chronic lymphocytic leukemia, myeloproliferative disorders, myelodysplastic syndromes, multiple myeloma, non-Hodgkin lymphoma, Hodgkin disease, aplastic anemia, pure red-cell aplasia, paroxysmal nocturnal hemoglobinuria, Fanconi anemia, thalassemia major, sickle cell anemia, severe combined immunodeficiency (SCID), Wiskott-Aldrich syndrome, hemophagocytic lymphohistiocytosis, inborn errors of metabolism, epidermolysis bullosa, severe congenital neutropenia, Shwachman-Diamond syndrome, Diamond-Blackfan anemia, and leukocyte adhesion deficiency.

In certain embodiments, the database comprises a data entry corresponding to each of a plurality of iPSC super donor cell lines, wherein the data entry for each super donor cell line comprises a set of characterized HLA loci corresponding to the super donor cell line.

In certain embodiments, each of the plurality of iPSC super donor cell lines can be used for treatment of a particular subject or particular group of subjects having matching HLA loci with lower risk of immune rejection by the particular subject or particular group of subjects.

In certain embodiments, the method further comprises determining the set of characterized HLA loci corresponding to each of the plurality of super donor cell lines by processing and analyzing one or more biological samples collected from each of one or more super donor individuals.

In certain embodiments, the step of determining the set of characterized HLA loci corresponding to each of the plurality of super donor cell lines comprises identifying a set of at least 3 HLA loci, wherein the at least 3 HLA loci are HLA-A, HLA-B, and HLA-DRB.

In certain embodiments, the step of determining the set of characterized HLA loci corresponding to each of the plurality of the super donor cell lines comprises identifying a set of at least 9 HLA loci, wherein the at least 9 HLA loci are HLA-A, HLA-B, HLA-C, HLA-DRB1, HLA-DRB3, HLA-DRB4, HLA-DRB5, HLA-DQB1, and HLA-DPB1.

In certain embodiments, the set of characterized HLA loci corresponding to each of the plurality of the super donor cell lines comprises at least 3 HLA loci selected from the group consisting of HLA-A, HLA-B, HLA-C, HLA-DRB1, HLA-DRB3, HLA-DRB4, HLA-DRB5, HLA-DQB1, and HLA-DPB1.

In certain embodiments, the set of characterized HLA loci corresponding to each of the plurality of the super donor cell lines are homozygous for HLA-A, HLA-B, and DRB-1.

In certain embodiments, the homozygous set of characterized HLA loci belong to a set of most-common HLA loci for a given population that matches a majority of the given population.

In certain embodiments, the homozygous set of characterized HLA loci comprise homozygous HLA loci in at least 3 major sites (e.g., or at least 4, or at least 5, or at least 6, or at least 7, or at least 8, or at least 9 major sites) wherein the major sites are members selected from the group consisting of HLA-A, HLA-B, HLA-C, HLA-DRB1, HLA-DRB3, HLA-DRB4, HLA-DRB5, HLA-DQB1, and HLA-DPB1.

In certain embodiments, the plurality of iPSC super donor cell lines match at least 70%(e.g., or at least 75%, or at least 80%, or at least 85%, or at least 90%, or at least 95%) of the population from which the particular subject originates.

In certain embodiments, the iPSC-derived CAR-T composition is produced using one of the plurality of iPSC super donor cell lines.

In certain embodiments, the method comprises exposing the iPSC super donor cell line used to produce the iPSC-derived CAR-T composition to culture media.

In certain embodiments, the iPSC-derived CAR-T composition comprises the plurality of compatible T cell lines or the plurality of iPSC super donor cell lines.

In certain embodiments, the compatible cells are engineered using CRISPR/Cas9 technology. In certain embodiments, the method comprises producing the iPSC-derived CAR-T composition using the engineered compatible cells.

In another aspect, the invention is directed to a composition of matter comprising an iPSC-derived CAR-T composition comprising one or more desired T cells, wherein the composition is produced by the method of any one of the aspects and embodiments described herein.

In certain embodiments, the iPSC-derived CAR-T composition comprises iPSCs and/or iPSC-derived cells derived from a biological sample of a particular subject. In certain embodiments, the iPSC-derived CAR-T composition is produced using the iPSCs and/or iPSC-derived cells derived from a biological sample of the subject.

In certain embodiments, the iPSC-derived CAR-T composition comprises compatible cells from a physical repository, wherein the compatible cells are identified as compatible to the particular subject or a particular group of subjects.

In certain embodiments, the compatible cells are identified as compatible with the particular subject or the particular group of subjects using an identification of cell type indicative of compatibility with the particular subject or particular group of subjects, wherein the identification of cell type indicative of compatibility comprises one or more of (i) to (iii): (i) an HLA match, (ii) an ABO blood type match, and (iii) an RHD blood group match having the same HLA loci, and/or ABO blood type, and/or RHD blood group as the particular subject or the particular group of subjects.

In another aspect, the invention is directed to a method of storing a iPSC-derived CAR-T composition tailored for treatment of a particular subject or particular group of subjects, said method comprising the steps of: (a) identifying, by a processor of a computing device, as compatible with the particular subject or particular group of subjects, one or more iPSC-derived CAR-T compositions derived using compatible cells corresponding to the one or more iPSCs and/or one or more iPSC-derived cells identified as compatible with the particular subject or particular group of subjects; (b) labelling, by a processor of a computing device, the one or more iPSC-derived CAR-T compositions with a label, wherein the label comprises information relating to the one or more iPSCs and/or one or more iPSC-derived cells, and a classification of the one or more iPSCs and/or one or more iPSC-derived cells the iPSC-derived CAR-T composition is derived from; and (c) storing, by a processor of a computing device, a database comprising a data entry corresponding to each label in a physical repository.

In certain embodiments, the classification of the one or more iPSCs and/or iPSC-derived cells comprises one or more of (i) to (iii): (i) HLA loci, (ii) ABO blood type, and (iii) RHD blood group.

In certain embodiments, the label is a physical label and/or digital label.

In certain embodiments, the label comprises information relating to one or more of (i) to (iii) as follows: (i) the one or more iPSCs and/or one or more iPSC-derived cells the iPSC-derived CAR-T composition is derived from; (ii) one or more HLA loci, and/or ABO blood type, and/or RHD blood group compatible with the labeled iPSC-derived CAR-T composition; and (iii) one or more iPSC-derived CAR-T compositions stored in the physical repository that are compatible with the particular subject or group of subjects, wherein the HLA loci, and/or ABO blood type, and/or RHD blood group of the one or more iPSC-derived CAR-T compositions are identical to or match the HLA loci, and/or ABO blood type, and/or RHD blood group of the one or more iPSCs and/or one or more iPSC-derived cells.

In another aspect, the invention is directed to a method of retrieving one or more produced, labeled and stored iPSC-derived CAR-T compositions derived using iPSCs and/or iPSC-derived cells, said method comprising the steps of: (a) identifying, by a processor of a computing device, as compatible with a particular subject or particular group of subjects, one or more iPSC-derived CAR-T compositions derived using one or more iPSCs and/or iPSC-derived cells identified as compatible with the particular subject or particular group of subjects; (b) retrieving from a physical repository the one or more compatible iPSC-derived CAR-T compositions corresponding to the one or more iPSCs and/or one or more iPSC-derived cells identified as compatible with the particular subject or particular group of subjects; and (c) updating, by a processor of a computing device, a database comprising data entries corresponding to the particular subject or particular group of subjects.

In certain embodiments, the retrieved iPSC-derived CAR-T compositions is administered as treatment to the particular subject or particular group of subjects.

In another aspect, the invention is directed to a method of administering an iPSC-derived CAR-T composition tailored for treatment of a particular subject or particular group of subjects, said method comprising the steps of: (a) identifying, by a processor of a computing device, the particular subject or particular group of subjects, as having low lymphocyte numbers or poor lymphocyte quality; (b) identifying, by the processor, as compatible with the particular subject or particular group of subjects, one or more iPSCs and/or one or more iPSC-derived cells; (c) retrieving compatible cells corresponding to the one or more iPSCs and/or one or more iPSC-derived cells identified as compatible with the particular subject or particular group of subjects; (d) producing the iPSC-derived CAR-T composition using the retrieved compatible cells; and (e) administering to the particular subject or particular group of subjects the iPSC-derived CAR-T composition.

In certain embodiments, step (c) comprises obtaining the compatible cells from a physical repository.

In certain embodiments, step (c) comprises retrieving the compatible cells using a processor-based query from a user, wherein the query comprises an identification of a cell type indicative of compatibility with the particular subject or particular group of subjects.

In certain embodiments, the identification of cell type indicative of compatibility with the particular subject or particular group of subjects comprises one or more of (i) to (iii): (i) an HLA match, (ii) an ABO blood type match, and (iii) an RHD blood group match.

In certain embodiments, step (b) comprises identifying, one or more stored and labeled iPSC-derived CAR-T compositions within the physical repository derived using one or more iPSCs and/or one or more iPSC-derived cells identified as compatible with the particular subject or particular group of subjects.

In certain embodiments, step (c) comprises retrieving, the one or more identified iPSC-derived CAR-T compositions corresponding to the one or more iPS cells and/or one or more iPSC-derived cells identified as compatible with the particular subject or particular group of subjects.

In certain embodiments, the compatible cells are engineered using CRISPR/Cas9 technology.

In certain embodiments, step (d) comprises producing the iPSC-derived CAR-T composition using the engineered compatible cells.

In another aspect, the invention is directed to a repository comprising a plurality of characterized iPSC-derived CAR-T compositions, wherein the repository is a biorepository for collecting, processing, storing, and/or distributing iPSC-derived CAR-T compositions.

In certain embodiments, the repository is in communication with one or more processors programmed for identifying, locating, and/or inventorying the biospecimens in the repository.

In certain embodiments, the repository is outfitted with hardware and/or robotics for automated sample handling.

In certain embodiments, each of the plurality of iPSC-derived CAR-T compositions is characterized by one or more of (i) to (iii): (i) an HLA match, (ii) an ABO blood type match, and (iii) an RHD blood group match.

In another aspect, the invention is directed to a method of using the repository of any one of the aspects and embodiments described herein for treatment of a particular subject or particular group of subjects, the method comprising: (a) identifying, by a processor of a computing device, as compatible with a particular subject or particular group of subjects, one or more iPSC-derived CAR-T compositions derived using one or more stem cells and/or one or more cell lines identified as compatible with the particular subject or particular group of subjects; and (b) retrieving, the one or more compatible iPSC-derived CAR-T compositions corresponding to the one or more stem cells and/or cell lines identified as compatible with the particular subject or particular group of subjects.

In another aspect, the invention is directed use of the repository of any one of the aspects and embodiments described herein for clinical procedures.

In certain embodiments, the clinical procedure is iPSC-derived CAR-T cell therapy.

In another aspect, the invention is directed use of the repository of any one of the aspects and embodiments described herein for pre-clinical studies.

In certain embodiments, the pre-clinical studies is a member selected from the group consisting of in vitro screens, in vivo screens, efficacy testing of medications, toxicity testing of medications, and testing for use in personalized medicine

Elements of embodiments involving one aspect of the invention (e.g., methods) can be applied in embodiments involving other aspects of the invention (e.g., systems), and vice versa.

### Definitions

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art.

“Sample” or “Biological Sample”: The term “sample” or “biological sample”, as used herein, refers to a biological sample obtained or derived from a source of interest, as described herein. In certain embodiments, a source of interest comprises an organism, such as a microbe, a plant, an animal or a human. In certain embodiments, a biological sample is or comprises biological tissue or fluid. In certain embodiments, a biological sample may be or comprise bone marrow; blood; blood cells; ascites; tissue or fine needle biopsy samples; cell-containing body fluids; free floating nucleic acids (e.g., cell free DNA); sputum; saliva; urine; cerebrospinal fluid, peritoneal fluid; pleural fluid; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In certain embodiments, a biological sample is or comprises cells obtained from an individual. In certain embodiments, obtained cells are or include cells from an individual from whom the sample is obtained. In certain embodiments, a sample is a “primary sample” obtained directly from a source of interest by any appropriate means. For example, in certain embodiments, a primary biological sample is obtained by methods selected from the group consisting of a swab, biopsy (e.g., fine needle aspiration or tissue biopsy), surgery, collection of body fluid (e.g., blood, lymph, feces etc.), etc. In certain embodiments, as will be clear from context, the term “sample” refers to a preparation that is obtained by processing (e.g., by removing one or more components of and/or by adding one or more agents to) a primary sample. For example, filtering using a semi-permeable membrane. Such a processed “sample” may comprise, for example nucleic acids or proteins extracted from a sample or obtained by subjecting a primary sample to techniques such as amplification or reverse transcription of mRNA, isolation and/or purification of certain components, etc.

“Genotyping data”: As used herein, the term “genotyping data” refers to data obtained from measurements of a genotype. In certain embodiments, genotyping data describes an individual's phenotype. Genotyping data may be measurements of particular genes (e.g., portions of an individual's genetic sequence, e.g., DNA sequence), SNPs, or variants of SNPs. In certain embodiments, genotyping data is obtained from a multi-gene panel. In certain embodiments, genotyping data is generated in response to a purchase or request by an individual. In certain embodiments, genotyping data comprises data for a portion of a genotype (e.g., of an individual). In certain embodiments, genotyping data comprises all available measurements of a genotype (e.g., of an individual).

“Partially un/differentiated”: As used herein, the term “partially un/differentiated” describes a biological cell that, like a state of stem cell, has a tendency to differentiate into a specific type of cell, but is already more specific than a stem cell and is pushed to differentiate into its “target” cell. For example, a difference between stem cells and progenitor cells is that stem cells can replicate indefinitely, whereas progenitor cells can divide only a limited number of times. An example of a partially un/differentiated cell is a progenitor cell.

“Subject” or “Individual”: As used herein, the term “subject” or “individual” refers to a human or other animal, or plant. In certain embodiments, subjects are humans and mammals (e.g., mice, rats, pigs, cats, dogs, horses, and primates). In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals are, for example, rodents (e.g., mice, rats, hamsters), rabbits, primates, or swine such as inbred pigs and the like.

“Bank”: As used herein, the term “bank” refers to a system, apparatus, or location where genetic material and/or biological sample is stored. Genetic material may be derived (e.g., extracted) from a biological sample provided by an individual to the organization that owns and/or operates the bank. In certain embodiments, biological samples are stored in a bank separate from a bank that stores genetic material extracted therefrom.

“Reserve”: As used herein, the term “reserve” refers to an amount of biological material (e.g., cells and/or cell lines) stored in a bank.

“Variant”: As used herein, the term “variant” refers to a specific variation of a specific SNP occurring in the genome of an organism. In certain embodiments, a variant is a specific combination of a first allele of a first copy of an individual's genetic material (e.g., corresponding to an individual's paternal DNA) and a second allele of a second copy of an individual's genetic material (e.g., corresponding to an individual's maternal DNA), as occurs in diploid organisms (e.g., humans).

“Cells” or “Cell lines” : As used herein, the term “cells” or “cells lines” refers to cells derived from human and/or non-human samples. In certain embodiments, cells can include in vitro cultured cells like iPSC-derived cells. In certain embodiments, cells can include cell lines. For example, cells can include iPSCs, and/or hematopoietic stem cells (HSCs), and/or blood progenitor cells, and/or mesenchymal stem cells (MSCs), and/or Retinal Pigment Epithelium (RPEs), and/or chondrocytes, and/or embryoid bodies, and/or any other iPSC-derived cells, and/or iPSC lines, and/or HSC lines, and/or blood progenitor cell lines, and/or MSCs lines, and/or RPE lines, and/or chondrocyte lines, and/or embryoid bodies of an iPSC line, and/or any other iPSC-derived cell lines. The cells and/or cell lines may or may not be immortalized.

Throughout the description, where compositions are described as having, including, or comprising specific components, or where methods are described as having, including, or comprising specific steps, it is contemplated that, additionally, there are compositions of the present invention that consist essentially of, or consist of, the recited components, and that there are methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim. Headers are provided for the convenience of the reader and are not intended to be limiting.

### DESCRIPTION OF THE DRAWINGS

The Drawings, which are comprised of at least the following Figures, is for illustration purposes only, not for limitation.

FIG. 1 shows an illustrative network environment 100 for use in the methods and systems described herein.

FIG. 2 shows an example of a computing device 200 and a mobile computing device 250 that can be used in the methods and systems described in this disclosure.

FIG. 3 is a block diagram of a method of manufacturing an induced pluripotent stem cell (iPSC)-derived chimeric antigen receptor T cell (CAR-T) composition tailored for treatment of a particular subject or particular group of subjects, according to an illustrative embodiment of the invention.

FIG. 4 is a block diagram of a method of storing an iPSC-derived CAR-T composition tailored for treatment of a particular subject or particular group of subjects, according to an illustrative embodiment of the invention.

FIG. 5 is a block diagram of a method of retrieving one or more produced, labeled and stored iPSC-derived CAR-T compositions from a physical repository, according to an illustrative embodiment of the invention.

FIG. 6 is a block diagram of a method of administering a iPSC-derived CAR-T composition tailored for treatment of a particular subject or particular group of subjects, according to an illustrative embodiment of the invention.

FIG. 7 is a block flow diagram of a method of manufacturing an induced pluripotent stem cell (iPSC)-derived chimeric antigen receptor T cell (CAR-T) composition tailored for treatment of a particular subject or particular group of subjects, according to an illustrative embodiment of the invention.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DETAILED DESCRIPTION

Presented herein are systems and methods of producing “universal” and/or “off-the-shelf” CAR-T compositions suitable for cancer (e.g., blood cancer) therapy to be administered to a specific individual and/or specific group of individuals. A CAR-T composition is a composition comprising one or more types of chimeric antigen receptor T cells (CAR-T). The iPS cells and/or cell lines, and any iPSC-derived CAR-T compositions derived therefrom, are identified as compatible with a specific individual or specific group of individuals using an identification of a cell type indicative of compatibility such as an HLA match and/or ABO blood match and/or RHD blood group match. The compatible iPS cells or cell lines are then retrieved from a managed HLA-indexed (and/or otherwise indexed) repository or are derived from a biological sample of a suitable donor. The retrieved compatible cells are then used to derive the “universal” and/or “off-the-shelf” iPSC-derived CAR-T composition, wherein the “universal” and/or “off-the-shelf” iPSC-derived CAR-T compositions are suitable for therapy of a specific individual and/or specific group of individuals.

The techniques described herein allow for the production of CAR-T compositions to be delivered to a specific individual or a specific group of individuals, thus enabling improved methods of cancer therapy, e.g., due to an enhanced compatibility of the specific individual or group of individuals with the cells from which the desired CAR-T composition is derived. Also, allogeneic iPS cells and/or cell lines that are compatible with a large portion of a specific population, e.g., super donors, can be prepared and stored in advance for large groups of individuals. These super donor-derived CAR-T compositions can then be made immediately available to people who need them, thus reducing production times of the iPSC-derived CAR-T compositions.

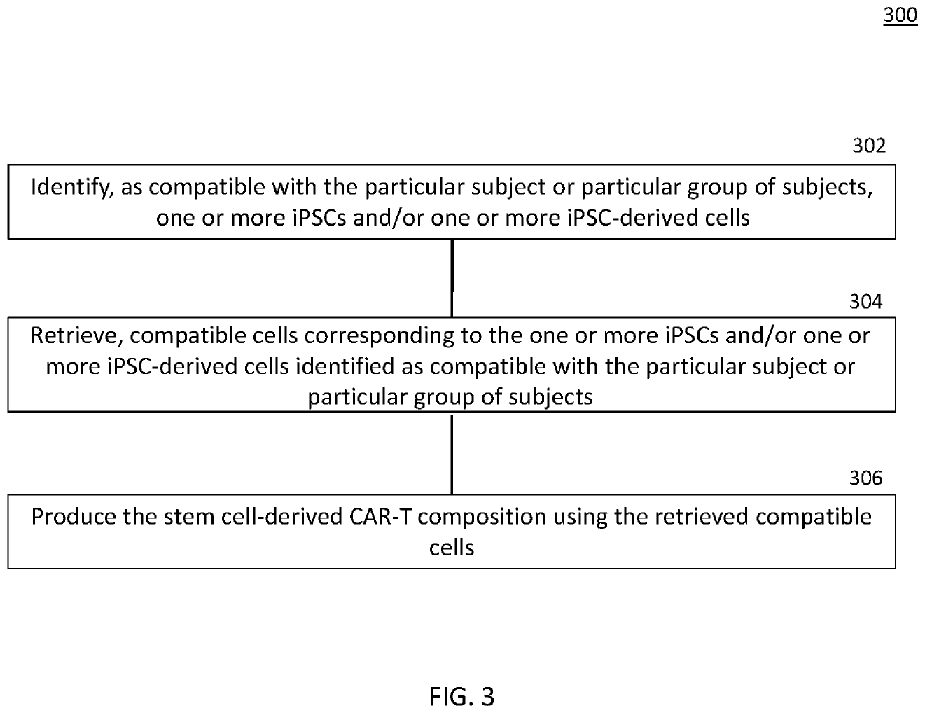

FIG. 3 is a block diagram showing a method 300 of manufacturing an iPSC-derived CAR-T composition, according to an illustrative embodiment of the invention. In step 302 the induced pluripotent stem (iPS) cells and/or iPSC-derived cells are identified as compatible with the particular subject or particular group of subjects. In certain embodiments, the iPS and/or iPSC-derived cells may belong to one or more cell types (e.g., HLA types), each of which is compatible with the particular subject or group of subjects. In certain embodiments one or more iPS cell lines and/or one or more iPSC-derived cell lines may also be identified, said cell lines being of one or more types (e.g., HLA types) each of which is compatible with the particular subject or group of subjects. In certain embodiments, the compatible cells and/or cell lines may be derived from the subject (e.g., autologous). In certain embodiments, the compatible cells and/or cell lines may be from an individual other than the subject (e.g., allogeneic). In step 304, the compatible cells corresponding to the one or more cells and/or cell lines identified as compatible with the particular subject or particular group of subjects are retrieved. The iPSC-derived CAR-T composition is then produced 306 using the retrieved compatible cells. In certain embodiments, the iPSC-derived CAR-T composition comprises compatible cells suitable for treatment of the subject.

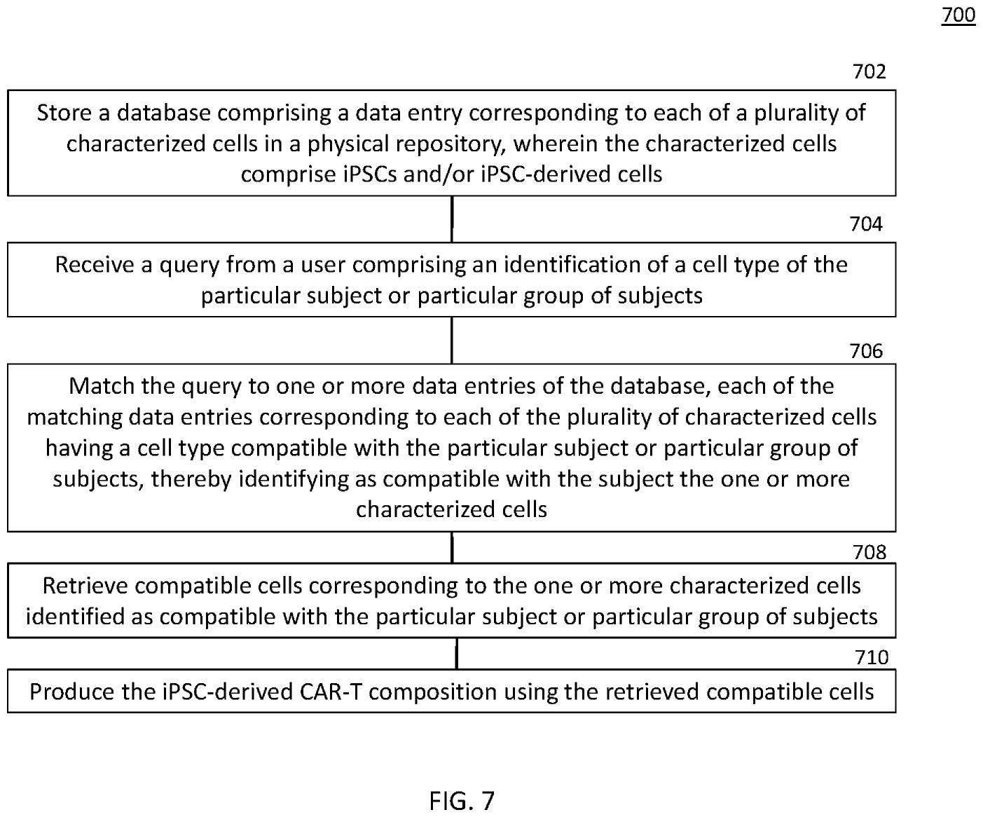

FIG. 7 is a block diagram showing a method 700 of manufacturing an induced pluripotent stem cell (iPSC)-derived CAR-T composition tailored for treatment of a particular subject or particular group of subjects. In step 702, using a processor of a computing device, a database comprising a data entry corresponding to each of a plurality of characterized cells in a physical repository are stored. In certain embodiments, the characterized cells comprise iPSCs and/or iPSC-derived cells (e.g., HSCs, MSCs, RPEs, chondrocytes, neurons, embryoid bodies and the like). In step 704, a query from a user comprising an identification of a cell type (e.g., HLA type, and/or ABO blood group, and/or RHD blood type) of the particular subject or particular group of subjects is received by the processor. In certain embodiments, the query may additionally comprise of ABO blood group, and/or RHD blood type. Then the query is matched (706) by the processor to one or more data entries of the database. Each of the matching data entries corresponds to each of the plurality of characterized cells (e.g., iPSCs, and/or iPSC-derived cells (e.g., HSCs, MSCs, RPEs, blood progenitors, chondrocytes, neurons, and embryoid bodies)), and/or iPSC lines, and/or iPSC-derived cell lines) having a cell type compatible with the particular subject or particular group of subjects. Each of the plurality of characterized cells corresponding to the matched data entries are identified as compatible with the particular subject or particular group of subjects. In step 708, the compatible cells corresponding to the one or more characterized cells identified as compatible with the particular subject or particular group of subjects are retrieved from a physical repository. The iPSC-derived CAR-T composition is then produced (710) using the retrieved compatible cells. In certain embodiments, the iPSC-derived CAR-T composition comprises compatible cells suitable for treatment of the subject.

In certain embodiments, the present disclosure is directed to a managed repository of CAR-T compositions derived from iPSCs, wherein each of the CAR-T compositions and/or iPSC lines has corresponding data comprising a set of characterized HLA loci, said corresponding data being stored in a searchable database for retrieval of one or more matching physical cell lines and/or CAR-T compositions upon query. The repository comprises a bank of iPSC-derived CAR-T compositions for each of a set of HLA types, for identification and provision of existing compatible iPSC-derived CAR-T compositions, and allogeneic cell lines suitable for deriving CAR-T compositions for treatment of cancer and/or other conditions.

### Human Leukocyte Antigen

The characterized iPS cells and/or cell lines stored in the repository are indexed using the Human Leukocyte Antigen (HLA). In certain embodiments, the iPS cells and/or cell lines and/or cell derived therefrom are characterized and indexed as super donor cell lines via HLA mapping (e.g., HLA typing and/or matching). In certain embodiments, multiple HLA loci may be characterized and indexed for each of the various iPS cells and/or cell lines and/or cells derived therefrom and/or compositions derived therefrom.

The HLAs in humans are major histocompatibility complex (WIC) proteins that function to regulate the immune system. HLA genes are highly polymorphic and may be broadly divided into Class I and Class II. For example, Class I in humans may be found on all nucleated cells and platelets. On the other hand, HLA Class II (constitutive expression), for example, may be restricted to specialized cells of the immune system (e.g., macrophages, B cells, etc.).

HLA Class I, for example, may include HLA-A, B, and C genes. In certain embodiments, HLA Class I may be co-dominantly expressed on the cell surface and may present peptides derived from internal cellular proteins to the T cell receptor of CD8 T cells. For example, these proteins may be involved in the immune response against intracellular parasites, viruses, and cancer.

In certain embodiments, HLA Class I may have a heterodimeric protein structure, with a polymorphic alpha chain and a common beta-2 microglobulin. In certain embodiments, the alpha chain may be composed of 3 extracellular domains: α1, α2, and α3.

HLA Class II, for example, may include DR, DQ and DP genes. In certain embodiments, HLA Class II may be co-dominantly expressed. In certain embodiments, HLA Class II may have a heterodimeric protein structure, with a polymorphic beta chain and a much less polymorphic alpha chain. In certain embodiments, both chains may be composed of two (2) extracellular domains (α1, α2, and β1, β2). For example, the α1 and β1 domains may create a peptide binding groove which presents processed peptides, from extracellular protein, to CD4+ T cells. In certain embodiments, HLA Class II may be involved in the immune response against extracellular infectious agents and non-self HLA molecules.

In certain embodiments, each HLA allele may be identified by letters indicating “locus” (e.g., A, B, C, DR, DQ, and DP) and individual specificity may be defined by a number following the locus (e.g., A1, B27, DR8, etc.). Specificities can be defined using antisera (antibodies). In certain embodiments, HLA specificities may also be determined using genetic analysis by identifying the presence/absence of the gene encoding the HLA protein. For example, Class II molecular specificities may be identified at the level of the gene encoding a particular chain (αor β).

### HLA Typing

The stem cells and/or stem cell lines (e.g., iPSCs) and/or cells derived therefrom and/or compositions derived therefrom stored in the physical repository may be characterized and indexed using various characteristics of the samples (e.g., cells). In certain embodiments, the stems cells and/or cell lines and/or cells derived therefrom and/or compositions derived therefrom may be characterized and indexed using HLA type. In certain embodiments, the stems cells and/or cell lines and/or cells derived therefrom and/or compositions derived therefrom may be characterized and indexed using ABO blood group. In certain embodiments, the stems cells and/or cell lines and/or cells derived therefrom and/or compositions derived therefrom may be characterized and indexed using RHD blood type. For example, the stems cells and/or cell lines and/or cells derived therefrom and/or compositions derived therefrom may be characterized and indexed in the physical repository using HLA type, and/or ABO blood group, and/or RHD type.

HLA typing or HLA matching is used to determine the HLA type of an individual. The HLA type of an individual comprises a pair of co-expressed haplotypes, each corresponding to a set of HLA genes (e.g., an HLA-A, an HLA-B, and an HLA-DR gene). In certain embodiments, genetic recombination and environmental factors result in linkage disequilibrium with respect to inheritance of HLA gene combinations. For example, certain combinations of HLA alleles (e.g., combinations of HLA-A, -B, and -DR genes) are favored, whereas other combinations do not exist.

HLA typing may be performed at a protein level but may also be performed at the DNA level, for example by amplifying the DNA via polymerase chain reaction (PCR), or other DNA identification and amplification technologies. For example, HLA typing may be performed using sequence specific oligonucleotides (SSO). In certain embodiments, SSO-based HLA typing may use generic primers to amplify large amounts of HLA alleles, for example, HLA-A, via PCR or other DNA amplification technologies. The dsDNA is separated into single strands and allowed to interact with the single strand specific oligonucleotide probes. In certain embodiments, such probes may be bound to a solid matrix. For example, the pattern of the bound probes may be used to determine the HLA type of the specimen. In certain embodiments, HLA typing may be performed using sequence specific primers (SSP). For example, in SSP-based HLA typing amplifies DNA that matches the primers. Antibodies may also been used for HLA typing, but may have the disadvantage of cross-reacting with multiple HLA epitopes (e.g. HLA-A2, A9 and A28).

### Applications of HLA Typing

The HLA type of a sample (e.g., cells, organs, and/or tissue) may be used in determining compatibility between organ donors and recipients. Samples which match the HLA type of a recipient (e.g., patient) are more likely to not illicit an immune response (e.g., rejection) after the sample is transplanted to the recipient. In certain embodiments, matching is performed on the basis of 3 or more loci on the HLA gene to prevent a strong immune response in the recipient post transplantation. In certain embodiments, at least 3 HLA loci are required to match between the donor and the recipient to prevent a strong immune response in the recipient post transplantation. In certain embodiments, at least 3, or at least 4, or at least 5, at least 6, or at least 7, or at least 8, or at least 9 major sites (e.g., loci) are required to match between the donor and the recipient to prevent a strong immune response in the recipient post transplantation.

Many registry donors have been tested by serological (e.g., HLA mapping using antigens) methods, though often without documentation regarding which antigens were tested. While the majority of hematopoietic progenitor cell transplant candidates have been tested by molecular (DNA-based) methodologies, the nomenclature of antigens (serology) and alleles (DNA) is in some cases not concordant. Thus, the characterized and indexed (e.g., HLA indexed (e.g., using standard nomenclature)) iPS cells and/or cell lines and/or cells derived therefrom and/or compositions derived therefrom, described herein, may be used to efficiently and accurately searched using the corresponding database to quickly find matching HLA samples for implantation. For example, the HLA indexed and matched iPS cells and/or cell lines and/or cells derived therefrom and/or compositions derived therefrom may be used in treatment of various diseases. In certain embodiments, these cells and/or cell lines may be used in the treatment cancer (e.g., leukemia, lymphoma, bone cancer, and the like). In certain embodiments, these cells and/or cell lines may be used in Hematopoietic stem cell transplantation.

The HLA-indexed repository may also be used for various purposes. For example, other clinical applications of HLA typing may include disease risk assessment, pharmacogenomics, immunotherapy, infectious disease vaccines, and tumor vaccines. In certain embodiments, the cells and/or cell lines stored and indexed in the repository may be used in cosmetic surgery, for example cartilage grafts. Long-term transplant and graft survival is correlated to the degree of HLA antigen mismatch for both solid organ and bone marrow transplant.

HLA matched cells and/or cell lines may also be used in the treatment of various diseases. Certain diseases may have a strong association with certain specific HLA types. For example, HLA associations with diseases include ankylosing spondylitis and acute anterior uveitis (HLA-B27); birdshot retinopathy (HLA-A29); Behget's Disease (HLA-B51); psoriasis (HLA-Cw6); celiac disease (HLA-DQ2,8); narcolepsy (HLA-DR15, DQ6); diabetes (HLA-DR3,4-DQ2,8); and rheumatoid arthritis (HLA-DR4). In certain embodiments, the data entries in the HLA database corresponding to specific samples (e.g., cells and/or cell lines in the physical repository) may incorporate information regarding their specific HLA types to recognize their strong associations with certain diseases.

HLA type may also be associated with allergy or hypersensitivity to a medication. For example, severe allergic or hypersensitivity reaction to drugs in Stevens-Johnson Syndrome (SJS) and toxic epidermal necrolysis (TEN) may be associated with HLA type. The physical repository of cells and/or cells lines and corresponding database may be used to identify allergies and sensitivities in the patients (e.g., sometimes unknown to the patient). In certain embodiments, HLA typing allows risk stratification of the patients. In certain embodiments, drugs that are associated with hypersensitivity reactions (e.g., antiepileptic agents, allopurinol, nevirapine, anti-inflammatories in oxicam family, and sulfonamides) may be studied using the cells and/or cell lines and/or cells derived therefrom stored in the repository. Further, these studies can be performed in vitro and/or ex vivo prior to implantation.

HLA typing may be used for vaccine development. The HLA-indexed cells and/or cell lines and/or cells derived therefrom and/or compositions derived therefrom described herein may be used to develop such vaccines. In certain embodiments, vaccines producing cellular immunity require peptide HLA binding. For example, vaccine trials use peptides binding to common HLA alleles. After proof-of-principal, trials may include peptides binding to other HLA alleles. In certain embodiments, cells with the common HLA allele, and cells with other HLA alleles may be selected from the back of stem cells and/or cell lines stored in the repository.

HLA typing can also be informative for compatibility of individuals. For example, studies have found that husbands and wives have fewer HLA matches than expected. The HLA genes (HLA-A, HLA-B, and HLA-DRB1) regulate the immune system, and thus determine the microbes that the immune system attacks. As a non-limiting example, the HLA genes therefore regulate a subject's smell by governing the non-human microbes associated with that subject and therefore can affect the attraction between subjects based on smell, among other things. Given the association between HLA type and long-term compatibility, it may be possible to predict the likelihood of companionship between two individuals. In some embodiments, the present disclosure teaches a method of querying and retrieving data entries of a database matching queried HLA loci for compatibility or companionship for a given subject with other individuals.

### HLA-Indexed Bank

The bank of IPS cells and/or cell lines and/or cells derived therefrom (e.g., HSCs and/or blood progenitors) and/or compositions derived therefrom is a comprehensive repository of in that it contains a variety of HLA types covering a significant proportion (e.g., at least 85%, at least 90%, or at least 95%) of a given population, indexed by HLA type and/or ABO group and/or RHD type. In certain embodiments, the HSC lines and/or blood progenitors in the bank (and/or the iPS cell lines and/or embryoid bodies from which the HSCs and/or blood progenitors are derived), may be characterized as super donor cell lines (e.g., via HLA mapping). Thus, it is possible to obviate the need for bone marrow registries and/or other donor registries, since suitable cells for transplantation may be quickly identified and made available to patients over a wide swath of a given population upon demand, without the difficult, time consuming process of identifying a matching blood marrow donor. Identification of a suitable cell line may include matching the patient's ABO blood type and/or RHD blood group to that of the HSC, blood progenitor cell, embryoid body, and/or iPSC line, in addition to HLA type.

The bank may provide access to reserves of immortalized iPSCs from which HSCs and/or blood progenitors can be derived—HSCs and/or blood progenitors may be prepared in advance for commonly-used/matched HLA types (e.g., HLA superdonors matching higher percentages of the population) so that cells are available immediately upon need. HSCs may also be produced for a particular patient upon identification of a matching iPSC line. Furthermore, in certain embodiments, reserves of embryoid bodies, corresponding to characterized iPSC lines, are stored in the bank. In certain embodiments, HLA superdonor lines are physically represented in the bank by embryoid bodies (characterized as HLA superdonor lines). These embryoid bodies may be used to make HSCs and/or blood progenitors.

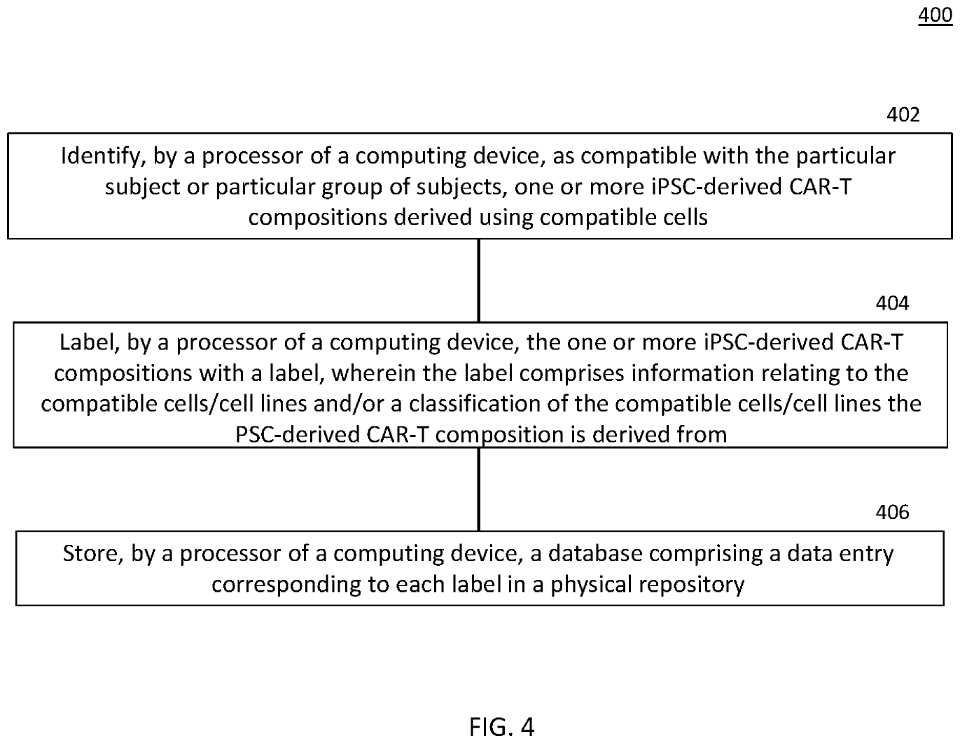

FIG. 4 is a block diagram showing a method 400 of storing an iPSC-derived CAR-T composition, according to an illustrative embodiment of the invention. In step 402, one or more iPSC-derived CAR-T compositions derived using compatible cells are identified, by a processor of a computing device, as compatible with the particular subject or particular group of subjects. In certain embodiments, the compatible cells correspond to one or more iPS (or iPSC-derived (e.g., MSC, HSC, RPE and the like)) cells and/or cell lines, said cells and/or cell lines being of one or more types (e.g., HLA type) each of which is identified as compatible with the particular subject or group of subjects. In step 404, the one or more iPSC-derived CAR-T compositions are labeled, by a processor of a computing device, with a label. In certain embodiments, the label may be a digital label, wherein the label comprises information relating to the iPS and/or iPSC-derived cell and/or cell line, and/or a classification of the iPS cell and/or cell line (e.g., HLA loci, and/or ABO blood type, and/or RHD blood group) the iPSC-derived CAR-T composition is derived from. The one or more labeled iPSC-derived CAR-T compositions are then stored (406), by a processor of a computing device, in a database comprising multiple data entries. One or more data entries in the database corresponds to each labeled iPSC-derived CAR-T compositions (e.g., or other labeled entities like cells, cell lines, other compositions and the like) stored in a physical repository.

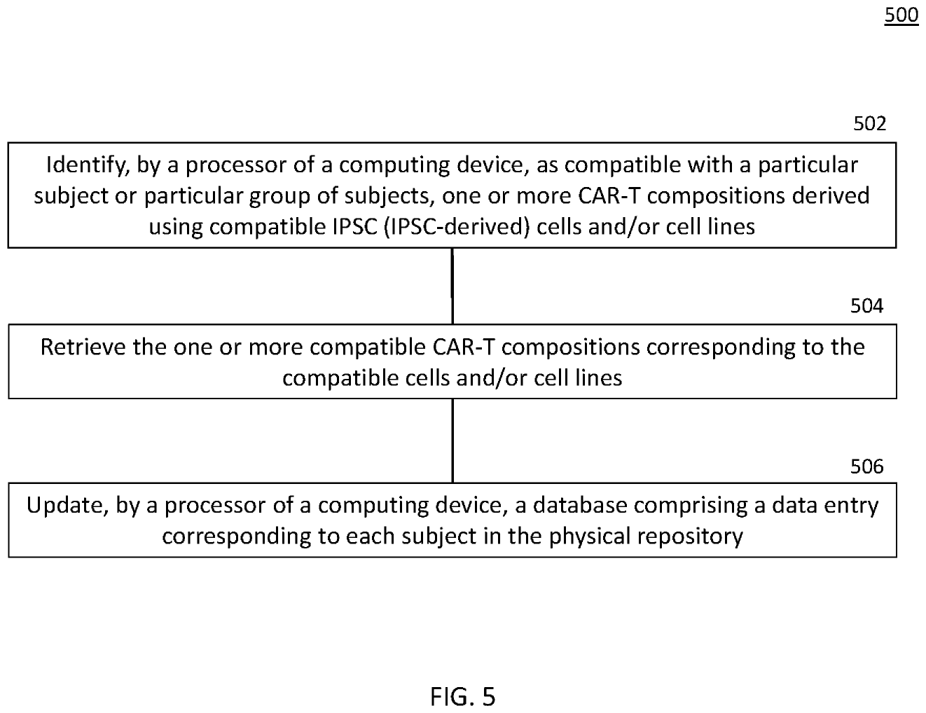

FIG. 5 is a block diagram showing a method 500 of retrieving one or more produced, labeled and stored iPSC-derived CAR-T compositions, according to an illustrative embodiment of the invention. In step 502, one or more iPSC-derived CAR-T compositions are identified, by a processor of a computing device, as compatible with a particular subject or particular group of subjects. The one or more iPSC-derived CAR-T compositions are derived using one or both of (i) and (ii) as follows: (i) one or iPS cells and/or iPSC-derived cells, said cells being of one or more types (e.g. HLA type) each of which is identified as compatible with the particular subject or group of subjects, and (ii) one or more iPS cell lines and/or one or more iPSC-derived cell lines, said cell lines being of one or more types each of which is identified as compatible with the particular subject or group of subjects. In step 504, the one or more compatible iPSC-derived CAR-T compositions corresponding to the one or more iPS and/or iPSC-derived cells and/or cell lines identified as compatible with the particular subject or particular group of subjects are retrieved (e.g., from the physical repository in which the one or more iPSC-derived CAR-T compositions are stored). The database data entry of each subject of the group of subjects is then updated (506), by a processor of a computing device. The update to the data entry corresponding to each subject may include identification information (e.g., label information) regarding the one or more iPSC-derived CAR-T compositions in the physical repository that each subject is compatible with.

Induced human pluripotent stem cells (iPSCs) can be generated from biological samples, such as blood samples. Depending on the conditions, in vitro iPSCs can retain their pluripotency or they can be directed to differentiate into a wide range of specialized cell types and tissues. Such cell types and tissues can be used for applications including replacement of diseased or damaged tissues in patients with conditions such as trauma, diabetes, degenerative neurological disorders, cardiovascular disease, and metabolic deficiencies.

As discussed in Taylor et al., Cell Stem Cell 11, Aug. 3, 2012, pp. 147-152, incorporated herein by reference, HLA-mismatched iPSCs can cause immunological rejection and therefore limit therapeutic potential. iPSCs derived directly from patients (autologous iPSCs) can result in matched HLA type and reduce risk of transplant rejection. However, generation of autologous iPSCs for individual patients is costly and time-consuming. Alternatively, allogeneic iPSC cell lines with HLA types that do not trigger strong reactions can be prepared and used for large groups of individuals.

The term “super donor” is a term used to describe HLA types that do not trigger strong rejection reactions. Such allogeneic (derived from donors other than the patient) iPSC lines can be made in advance and can be ready for use when needed. Fewer allogeneic lines are needed to serve a population. iPSCs can be obtained from healthy volunteer donors of blood group O that are selected to maximize the opportunity for HLA matching. Clinical grade iPSC lines can be expanded and differentiated for use in a large number of subjects. Nakajima et al., Stem Cells 25, 2007, pp. 983-985, incorporated by reference herein, discusses HLA matching estimations in a hypothetical bank of human embryonic stem cell lines in the Japanese population, and calculated that a large proportion of patients were able to find at least one HLA matched donor at three loci of HLA-A, HLA-B, and HLA-DR for transplantation therapy. Because the iPSC lines are characterized by HLA type, an iPSC line can be identified as suitable for a given patient (or multiple patients) with a compatible HLA type, with low, reduced, or zero chance of a compatible cell-derived CAR-T composition rejection. In certain embodiments, the bank of iPSCs is comprehensive in that it contains a variety of HLA types covering a significant proportion (e.g., at least 85%, at least 90%, or at least 95%) of a given population. In certain embodiments, the CAR-T compositions in the bank and/or the iPS cell lines from which the CAR-T compositions are derived, are characterized as super donor cell lines (e.g., via HLA mapping). Thus, suitable cells, cell lines and/or iPSC-derived CAR-T compositions for treatment may be quickly identified and made available to patients over a wide swath of a given population upon demand, without the difficult, time consuming process of identifying a matching donor. Identification of a suitable cell line and/or iPSC-derived CAR-T composition may include matching the patient's ABO blood type and/or RHD blood group to that of the iPSC line and/or CAR-T composition in addition to HLA type.

The bank may provide access to reserves of immortalized iPSCs from which CAR-T compositions can be derived. In certain embodiments, MSCs, HSCs, blood progenitors, embryoid bodies and/or other immunogenic cell types may be prepared in advance for commonly-used/matched HLA types (e.g., HLA super donors matching higher percentages of the population) so that CAR-T compositions are available immediately upon need. CAR-T compositions may also be produced for a particular patient or a group of particular patients upon identification of a matching iPSC line. Furthermore, in certain embodiments, reserves of embryoid bodies, corresponding to characterized iPSC lines, are stored in the bank. In certain embodiments, HLA super donor lines are physically represented in the bank by embryoid bodies (characterized as HLA super donor lines). These embryoid bodies may be used to make immunogenic cells (e.g., T cells) that are used to formulate the CAR-T compositions.

The characterized iPSCs and/or embryoid bodies comprising embryonic stem cells (e.g., undifferentiated pluripotent cells) can be differentiated into immune cells like macrophages, B lymphocytes, T lymphocytes, etc. in the presence of appropriate culture media. In certain embodiments, the characterized cell types contained in the physical bank include any one or more of the following: iPSCs, embryoid bodies, MSCs, HSCs, blood progenitor cells, and/or mature hematopoietic cells.

Matching HLA type may involve, for example, querying and retrieving data entries of a database matching queried HLA loci. In certain embodiments, this comprises receiving, by a processor of a computing device (e.g., a server), a data entry for an individual for which a matching iPSC and/or HSC line, and/or iPSC-derived CAR-T composition is desired, the data entry comprising a set of characterized HLA loci corresponding to the individual [e.g., identification (e.g., by processing and analyzing (e.g., by serology, by PCR) samples from the individual (e.g., blood samples)) of each of a set of at least 3 given loci (e.g., HLA-A, HLA-B, and HLA-DRB (e.g., HLA-DRB1)), e.g., at least 9 given loci (e.g., HLA-A, HLA-B, HLA-C, HLA-DRB1, HLA-DRB3, HLA-DRB4, HLA-DRB5, HLA-DQB1, HLA-DPB1), e.g., at least 3, 4, 5, 6, 7, 8, or 9 members selected from this group of nine loci]; and retrieving, by the processor, one or more data entries of a database representative of cells (e.g., iPS cells in the physical repository and/or embryoid bodies from a cell line derived from iPSCs), and/or CAR-T compositions matching (e.g., exactly matching, partially matching, identified as compatible with (e.g., compatible HLA types), etc.) the queried HLA loci (e.g., determining the corresponding bar code or other identifier for the iPSCs and/or embryoid bodies corresponding to the data entry, thereby allowing retrieval of desired stem cells and/or CAR-T compositions from the repository and/or retrieval of identifying information corresponding to a desired iPSC cell line matching the queried HLA loci. iPSC-derived CAR-T compositions may be produced from immortalized iPSC lines at will and made available for ready access when needed—no additional harvesting of samples are required to produce additional iPSC-derived CAR-T compositions.

The repository/bank of cells and compositions may comprise a storage system comprising an insulated container equipped with environmental control system (for control of temperature, humidity, pressure, and the like) suitable to store cells (e.g., iPSCs), and CAR-T compositions (e.g., derived from iPSCs) for a period of time. The repository/bank may also include one or more processors (e.g., of a server) and/or related software to manage inventory, as well as a sample location system and/or retrieval system for identification/retrieval of cells and/or specific CAR-T compositions from a matched cell line. iPSCs may be produced from blood samples (or other biological substance sample, e.g., saliva, serum, tissue, cheek cells, cells collected via a buccal swab, urine, and/or hair), then labeled (physically and/or digitally), logged in an inventory database, and stored in the repository for ongoing and/or future use. The iPSC-derived cells and/or CAR-T compositions may also be labeled (physically and/or digitally), logged in the inventory database, and stored in the repository for ongoing and/or future use.

The repository/bank of cells may be used in systems and methods for regeneration, treatment of subjects in need of cancer (e.g., blood cancer) therapy. For example, the repository/bank of cells comprise iPSCs and/or embryoid bodies corresponding to/produced from iPSC lines, wherein CAR-T compositions are derived from/produced from the iPSCs and/or embryoid bodies, and the CAR-T compositions are administered to subjects at risk of or having a disease and/or condition, such as any of the following: acute myeloid leukemia, acute lymphoblastic leukemia, chronic myeloid leukemia, chronic lymphocytic leukemia, myeloproliferative disorders, myelodysplastic syndromes, multiple myeloma, non-Hodgkin lymphoma, Hodgkin disease, aplastic anemia, pure red-cell aplasia, paroxysmal nocturnal hemoglobinuria, Fanconi anemia, thalassemia major, sickle cell anemia, severe combined immunodeficiency (SCID), Wiskott-Aldrich syndrome, hemophagocytic lymphohistiocytosis, inborn errors of metabolism, epidermolysis bullosa, severe congenital neutropenia, Shwachman-Diamond syndrome, Diamond-Blackfan anemia, and leukocyte adhesion deficiency.

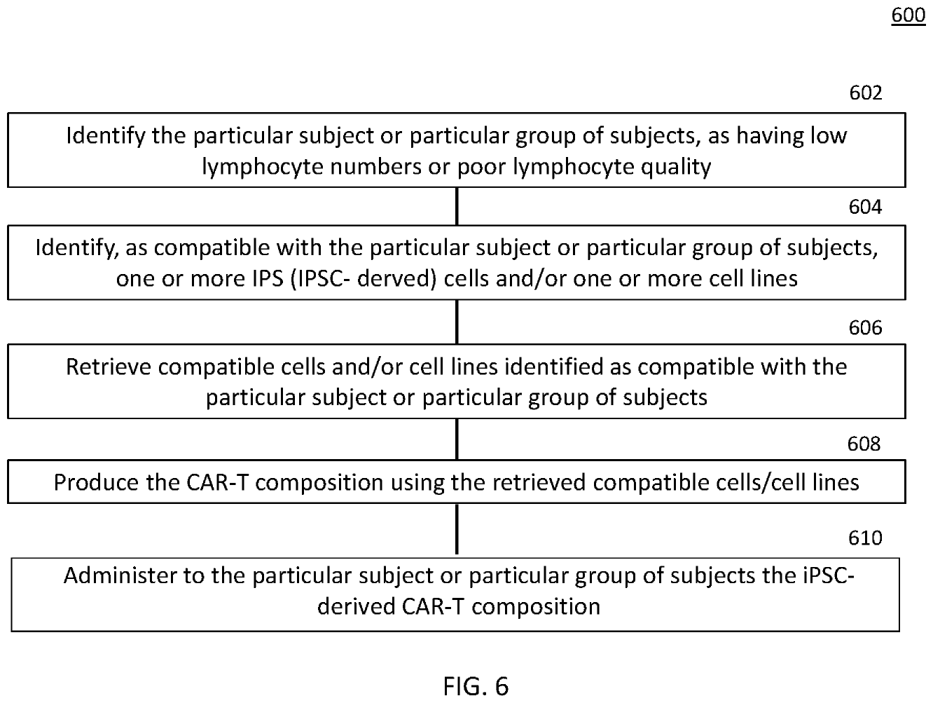

For example, FIG. 6 is a block diagram showing a method 600 of administering an iPSC-derived CAR-T composition tailored for treatment of a particular subject or particular group of subjects, according to an illustrative embodiment of the invention. In step 602, the particular subject or particular group of subjects having low lymphocyte numbers or poor lymphocyte quality is/are identified. In second step 604, one or both of (i) and (ii) as follows: (i) one or more induced pluripotent stem (iPS) cells and/or iPSC-derived cells, said cells being of one or more types each of which is compatible with the particular subject or group of subjects, and (ii) one or more iPS cell lines and/or one or more iPSC-derived cell lines, said cell lines being of one or more types each of which is compatible with the particular subject or group of subjects, are identified as compatible with the particular subject or particular group of subjects. Following identification, in step 606, the compatible cells corresponding to the iPS and/or iPSC-derived cells and/or cell lines identified as compatible with the particular subject or particular group of subjects are retrieved (e.g., from a physical repository). The iPSC-derived CAR-T composition is then produced (608) using the retrieved compatible cells. The iPSC-derived CAR-T composition produced is engineered and/or selected such that it offsets the deficiency in the particular subject or particular group of subjects. The iPSC-derived CAR-T composition is then administered (610) to the particular subject or particular group of subjects.

Generation and Differentiation Protocols for Immortalized iPSCs

Induced pluripotent stem cell (iPSC) generation protocols are described, for example, at https://www.thermofisher.com/us/en/home/references/protocols/cell-culture/stem-cell-protocols/ipsc-protocols.html, the contents of which is hereby incorporated by reference in its entirety. Induced pluripotent stem cell (iPSC) generation and differentiation protocols are described, for example, at http://www.sigmaaldrich.com/life-science/stem-cell-biology/ipsc/ipsc-protocols.html, the contents of which is hereby incorporated by reference in its entirety. Differentiation of iPSCs can be found, for example, in “Induction of Pluripotent Stem Cells from Adult Human Fibroblasts by Defined Factors”; Takahashi K., Tanabe K., Ohnuki M., Narita M., Ichisaka T., Tomoda K., Yamanaka S.; Cell Vol. 131, 861-872, November 2007″, the contents of which is hereby incorporated by reference in its entirety.

Engineered induced pluripotent stem cell (iPSC)-derived T lymphocytes (or T cells) have been used to help produce “off-the-shelf” histocompatible cell therapy products for cancer treatment. See, for example, “New Cell Sources for T Cell Engineering and Adoptive Immunotherapy”, Cell Stem Cell. 2015 April; 16(4); 357-366; the contents of each of which are incorporated herein by reference. Furthermore, in recent years, there have been significant advances in the production of iPSCs from cells collected from a biological sample of a subject (e.g., blood cells). For example, iPSCs can be made by inserting copies of stem cell-associated genes—e.g., Oct 3/4, Sox 2, Klf4, and c-Myc (or Oct 3/4, Sox 2, Nanog, and Lin28)—into cells collected from the biological sample using viral vectors. See, for example, K. Okita, T. Ichisaka, and S. Yamanaka, “Generation of germline-competent induced pluripotent stem cells,” Nature, vol. 448, no. 7151, pp. 313-317, 2007; K. Okita, Y. Matsumura, Y. Sato et al., “A more efficient method to generate integration-free human iPS cells,” Nature Methods, vol. 8, no. 5, pp. 409-412, 2011; Themeli et al., “Generation of tumor-targeted human T lymphocytes from induced pluripotent stem cells for cancer therapy”, Nature Biotechnology, 31, 928-933 (published online 11 Aug. 2013); the contents of each of which are incorporate herein by reference.

Storage of Immortalized iPSCs

Repositories (290) (e.g., cell repositories, e.g., nucleic acid repositories) for storing biological sample material (e.g., cells, e.g., nucleic acids) can include liquid nitrogen storage tanks and/or other freezer systems. Liquid nitrogen tanks provide temperature (e.g., about −195° C.) and/or humidity control, and can be used to store, for example, immortalized cell lines (e.g., immortalized iPSCs) over a long period of time. Alternatively, biological material (e.g., nucleic acids) can be stored in freezer systems at higher temperatures (e.g., from about −80° C. to about −20° C.). Additional equipment, backup systems, software/inventory control systems, sample location systems, automated sample retrieval, etc. can be used for storage and/or maintenance of the biological sample material stored in the repositories. The described setup allows for backup systems (e.g., additional repositories) to be used if a given tank and/or freezer temperature control system and/or humidity control system malfunctions.

Moreover, the provided systems and methods can record and track, via a graphical user interface, biological samples (and biological material extracted therefrom) used to generate genotyping data, for example, as described in U.S. Application No. 62/485,778, entitled “Chain Of Custody For Biological Samples And Biological Material Used In Genotyping Tests” and filed on Apr. 14, 2017, U.S. application Ser. No. 15/846, 659 entitled “Chain Of Custody For Biological Samples And Biological Material Used In Genotyping Tests” filed on Dec. 19, 2017, and International Application No. PCT/US17/67272 entitled “Chain of Custody for Biological Samples and Biological Material Used in Genotyping Tests” filed on Dec. 19, 2017, the contents of which are hereby incorporated by reference in their entirety.

For example, as biological samples are processed in several stages to extract biological material and perform genotyping tests, IDs are assigned to biological sample material for individuals as well as well plates used during processing of the biological sample material in order to organize the samples and the tests. Biological sample materials are assigned to well plates for use in extracting biological material. Biological sample material is assigned to genotyping plates for use in performing genotyping tests. By associating IDs corresponding to biological sample material with IDs for well plates or genotyping plates, respectively, a user can track which extractions and/or tests need to be performed as well as record which biological samples have been received or genotyping plates analyzed via a graphical user interface.

### Illustrative Computer Network Environment

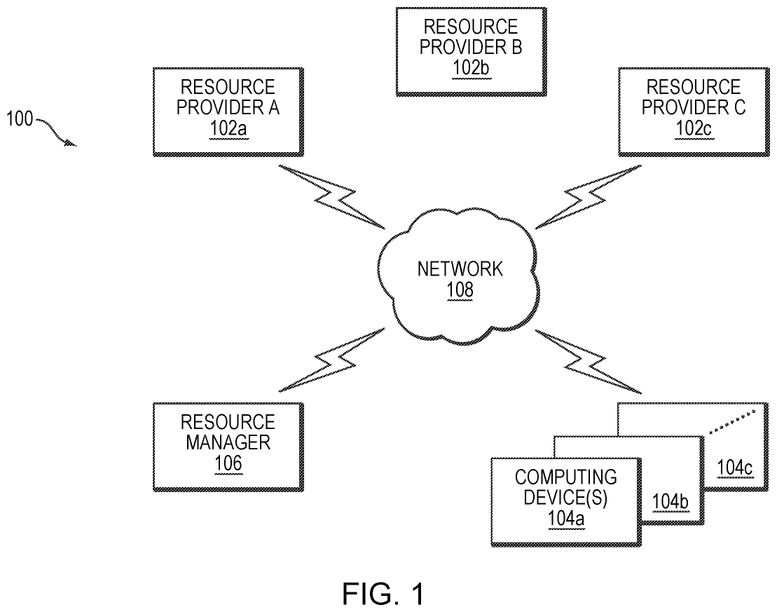

FIG. 1 shows an illustrative network environment 100 for use in the methods and systems described herein. In brief overview, referring now to FIG. 1, a block diagram of an exemplary cloud computing environment 100 is shown and described. The cloud computing environment 100 may include one or more resource providers 102a, 102b, 102c (collectively, 102). Each resource provider 102 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 102 may be connected to any other resource provider 102 in the cloud computing environment 100. In some implementations, the resource providers 102 may be connected over a computer network 108. Each resource provider 102 may be connected to one or more computing device 104a, 104b, 104c (collectively, 104), over the computer network 108.

The cloud computing environment 100 may include a resource manager 106. The resource manager 106 may be connected to the resource providers 102 and the computing devices 104 over the computer network 108. In some implementations, the resource manager 106 may facilitate the provision of computing resources by one or more resource providers 102 to one or more computing devices 104. The resource manager 106 may receive a request for a computing resource from a particular computing device 104. The resource manager 106 may identify one or more resource providers 102 capable of providing the computing resource requested by the computing device 104. The resource manager 106 may select a resource provider 102 to provide the computing resource. The resource manager 106 may facilitate a connection between the resource provider 102 and a particular computing device 104. In some implementations, the resource manager 106 may establish a connection between a particular resource provider 102 and a particular computing device 104. In some implementations, the resource manager 106 may redirect a particular computing device 104 to a particular resource provider 102 with the requested computing resource.

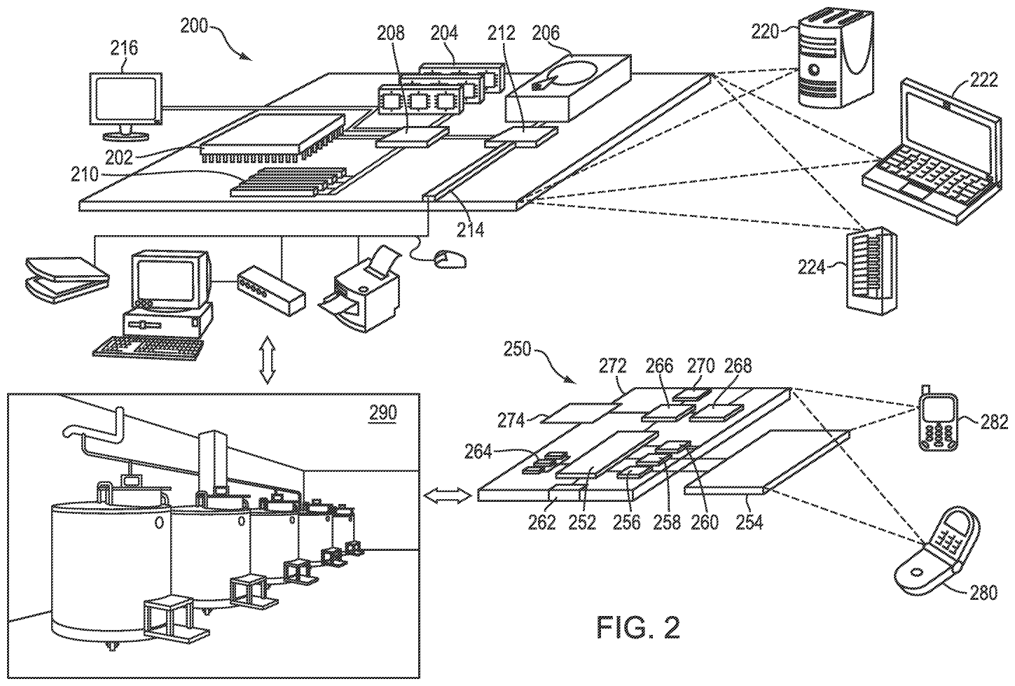

FIG. 2 shows an example of a computing device 200 and a mobile computing device 250 that can be used in the methods and systems described in this disclosure. The computing device 200 is intended to represent various forms of digital computers, such as laptops, desktops, workstations, personal digital assistants, servers, blade servers, mainframes, and other appropriate computers. The mobile computing device 250 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 200 includes a processor 202, a memory 204, a storage device 206, a high-speed interface 208 connecting to the memory 204 and multiple high-speed expansion ports 210, and a low-speed interface 212 connecting to a low-speed expansion port 214 and the storage device 206. Each of the processor 202, the memory 204, the storage device 206, the high-speed interface 208, the high-speed expansion ports 210, and the low-speed interface 212, are interconnected using various busses, and may be mounted on a common motherboard or in other manners as appropriate. The processor 202 can process instructions for execution within the computing device 200, including instructions stored in the memory 204 or on the storage device 206 to display graphical information for a GUI on an external input/output device, such as a display 216 coupled to the high-speed interface 208. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 204 stores information within the computing device 200. In some implementations, the memory 204 is a volatile memory unit or units. In some implementations, the memory 204 is a non-volatile memory unit or units. The memory 204 may also be another form of computer-readable medium, such as a magnetic or optical disk.

The storage device 206 is capable of providing mass storage for the computing device 200. In some implementations, the storage device 206 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 202), perform one or more methods, such as those described above. The instructions can also be stored by one or more storage devices such as computer- or machine-readable mediums (for example, the memory 204, the storage device 206, or memory on the processor 202).

The high-speed interface 208 manages bandwidth-intensive operations for the computing device 200, while the low-speed interface 212 manages lower bandwidth-intensive operations. Such allocation of functions is an example only. In some implementations, the high-speed interface 208 is coupled to the memory 204, the display 216 (e.g., through a graphics processor or accelerator), and to the high-speed expansion ports 210, which may accept various expansion cards (not shown). In the implementation, the low-speed interface 212 is coupled to the storage device 206 and the low-speed expansion port 214. The low-speed expansion port 214, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 200 may be implemented in a number of different forms, as shown in the figure. For example, it may be implemented as a standard server 220, or multiple times in a group of such servers. In addition, it may be implemented in a personal computer such as a laptop computer 222. It may also be implemented as part of a rack server system 224. Alternatively, components from the computing device 200 may be combined with other components in a mobile device (not shown), such as a mobile computing device 250. Each of such devices may contain one or more of the computing device 200 and the mobile computing device 250, and an entire system may be made up of multiple computing devices communicating with each other.

The mobile computing device 250 includes a processor 252, a memory 264, an input/output device such as a display 254, a communication interface 266, and a transceiver 268, among other components. The mobile computing device 250 may also be provided with a storage device, such as a micro-drive or other device, to provide additional storage. Each of the processor 252, the memory 264, the display 254, the communication interface 266, and the transceiver 268, are interconnected using various buses, and several of the components may be mounted on a common motherboard or in other manners as appropriate.

The processor 252 can execute instructions within the mobile computing device 250, including instructions stored in the memory 264. The processor 252 may be implemented as a chipset of chips that include separate and multiple analog and digital processors. The processor 252 may provide, for example, for coordination of the other components of the mobile computing device 250, such as control of user interfaces, applications run by the mobile computing device 250, and wireless communication by the mobile computing device 250.

The processor 252 may communicate with a user through a control interface 258 and a display interface 256 coupled to the display 254. The display 254 may be, for example, a TFT (Thin-Film-Transistor Liquid Crystal Display) display or an OLED (Organic Light Emitting Diode) display, or other appropriate display technology. The display interface 256 may comprise appropriate circuitry for driving the display 254 to present graphical and other information to a user. The control interface 258 may receive commands from a user and convert them for submission to the processor 252. In addition, an external interface 262 may provide communication with the processor 252, so as to enable near area communication of the mobile computing device 250 with other devices. The external interface 262 may provide, for example, for wired communication in some implementations, or for wireless communication in other implementations, and multiple interfaces may also be used.

The memory 264 stores information within the mobile computing device 250. The memory 264 can be implemented as one or more of a computer-readable medium or media, a volatile memory unit or units, or a non-volatile memory unit or units. An expansion memory 274 may also be provided and connected to the mobile computing device 250 through an expansion interface 272, which may include, for example, a SIMM (Single In Line Memory Module) card interface. The expansion memory 274 may provide extra storage space for the mobile computing device 250, or may also store applications or other information for the mobile computing device 250. Specifically, the expansion memory 274 may include instructions to carry out or supplement the processes described above, and may include secure information also. Thus, for example, the expansion memory 274 may be provided as a security module for the mobile computing device 250, and may be programmed with instructions that permit secure use of the mobile computing device 250. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier and, when executed by one or more processing devices (for example, processor 252), perform one or more methods, such as those described above. The instructions can also be stored by one or more storage devices, such as one or more computer- or machine-readable mediums (for example, the memory 264, the expansion memory 274, or memory on the processor 252). In some implementations, the instructions can be received in a propagated signal, for example, over the transceiver 268 or the external interface 262.

The mobile computing device 250 may communicate wirelessly through the communication interface 266, which may include digital signal processing circuitry where necessary. The communication interface 266 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 268 using a radio-frequency. In addition, short-range communication may occur, such as using a Bluetooth®, Wi-Fi™, or other such transceiver (not shown). In addition, a GPS (Global Positioning System) receiver module 270 may provide additional navigation- and location-related wireless data to the mobile computing device 250, which may be used as appropriate by applications running on the mobile computing device 250.

The mobile computing device 250 may also communicate audibly using an audio codec 260, which may receive spoken information from a user and convert it to usable digital information. The audio codec 260 may likewise generate audible sound for a user, such as through a speaker, e.g., in a handset of the mobile computing device 250. Such sound may include sound from voice telephone calls, may include recorded sound (e.g., voice messages, music files, etc.) and may also include sound generated by applications operating on the mobile computing device 250.

The mobile computing device 250 may be implemented in a number of different forms, as shown in the figure. For example, it may be implemented as a cellular telephone 280. It may also be implemented as part of a smart-phone 282, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In certain embodiments, the system comprises a physical biorepository 290 (comprising one or more cell storage containers) in communication with any of the computer system arrangements of FIG. 1 or 2.

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim. Headers are provided for the convenience of the reader and are not intended to be limiting with respect to the claimed subject matter.

Documents are incorporated herein by reference as noted. Where there is any discrepancy in the meaning of a particular term, the meaning provided in the Definition section above is controlling.

Certain embodiments of the present invention are described herein. It is, however, expressly noted that the present invention is not limited to these embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description. Therefore, the disclosure should not be limited to certain implementations, but rather should be limited only by the spirit and scope of the claims.

### Equivalents

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of manufacturing an induced pluripotent stem cell (iPSC)-derived chimeric antigen receptor T cell (CAR-T) composition tailored for treatment of a particular subject or particular group of subjects, said method comprising the steps of:
(a) identifying, as compatible with the particular subject or particular group of subjects, one or more iPSCs and/or one or more iPSC-derived cells;
(b) retrieving compatible cells corresponding to the one or more iPSCs and/or one or more iPSC-derived cells identified as compatible with the particular subject or particular group of subjects; and
(c) producing the iPSC-derived CAR-T composition using the retrieved compatible cells.

2. The method of claim 1, wherein the compatible cells comprise one or more members selected from the group consisting of iPSCs, stem cells, blood progenitors, iPSCs, hematopoietic stem cells (HSCs), mesenchymal stem cells (MSCs), Retinal Pigment Epithelium (RPE), and embryoid bodies.

3. The method of claim 1 or claim 2, wherein step (b) comprises deriving the compatible cells from a biological sample of a donor.

4. The method of claim 2, wherein the donor is not the particular subject or a member of the particular group of subjects.

5. The method of any one of the preceding claims, wherein step (b) comprises obtaining the compatible cells from a physical repository.

6. The method of any one of the preceding claims, wherein step (b) comprises retrieving, by a processor of a computing device, one or more data entries corresponding to the compatible cells using a processor-based query from a user, wherein the query comprises an identification of a cell type indicative of compatibility with the subject.

7. The method of claim 6, wherein the identification of cell type indicative of compatibility with the particular subject or particular group of subjects comprises one or more of (i) to (iii): (i) an HLA match, (ii) an ABO blood type match, and (iii) an RHD blood group match.

8. The method of any one of the preceding claims, wherein the iPSC-derived CAR-T composition comprises the retrieved compatible cells.

9. The method of any one of the preceding claims, comprising producing T cells from the one or more iPSCs, and/or one or more iPSC-derived cells identified as compatible with the particular subject or particular group of subjects.

10. A method of manufacturing an induced pluripotent stem cell (iPSC)-derived chimeric antigen receptor T cell (CAR-T) composition tailored for treatment of a particular subject or particular group of subjects, said method comprising the steps of:
(a) storing, by a processor of a computing device, a database comprising a data entry corresponding to each of a plurality of characterized cells in a physical repository, wherein the characterized cells comprise iPSCs and/or iPSC-derived cells;
(b) receiving, by the processor, a query from a user comprising an identification of a cell type of the particular subject or particular group of subjects;
(c) matching, by the processor, the query to one or more data entries of the database, each of the matching data entries corresponding to each of the plurality of characterized cells having a cell type compatible with the particular subject or particular group of subjects, thereby identifying as compatible with the subject the one or more characterized cells;
(d) retrieving, from a physical repository, compatible cells corresponding to the one or more characterized cells identified as compatible with the particular subject or particular group of subjects; and
(e) producing the iPSC-derived CAR-T composition using the retrieved compatible cells.

11. The method of claim 11, wherein:
the data entry corresponding to each of the plurality of characterized cells comprises a set of characterized HLA loci corresponding to the cell,
the query comprises a set of queried HLA loci for the particular subject or the particular group of subjects, and
the one or more matched data entries of the database are each representative of one or more characterized compatible cells matching the queried HLA loci.

12. The method of claim 11, wherein the plurality of characterized cells in the physical repository are immortalized.

13. The method of claim 11 or claim 12, wherein the set of characterized HLA loci corresponding to each of the plurality of characterized cells comprises a set of at least 3 HLA loci, wherein the at least 3 HLA loci are HLA-A, HLA-B, and HLA-DRB.

14. The method of claim 11 or claim 12, wherein the set of characterized HLA loci corresponding to each of the plurality of characterized cells comprises a set of at least 9 given loci, wherein the at least 9 given loci are HLA-A, HLA-B, HLA-C, HLA-DRB1, HLA-DRB3, HLA-DRB4, HLA-DRB5, HLA-DQB1, HLA-DPB1.

15. The method of claim 13, wherein the set of characterized HLA loci corresponding to each of the plurality of characterized cells comprises at least 3 given loci selected from the group consisting of HLA-A, HLA-B, HLA-C, HLA-DRB1, HLA-DRB3, HLA-DRB4, HLA-DRB5, HLA-DQB1, and HLA-DPB1.

16. The method of any one of claims 11 to 15, wherein each of the one or more matching data entries of the database exactly match or partially match the set of queried HLA loci for the particular subject or the particular group of subjects.

17. The method of any one of claims 11 to 16, comprising determining a corresponding bar code or other identifier for the one or more characterized compatible cells corresponding to each of the retrieved matching data entries, thereby allowing retrieval of desired cells from the physical repository and/or retrieval of identifying information corresponding to the one or more characterized compatible cells matching the queried HLA loci.

18. The method of any one of claims 11 to 17, wherein the data entry for each of the plurality of characterized cells further comprises ABO blood type and the query further comprises ABO blood type, and wherein the one or more matching data entries of the database representative of the one or more characterized compatible cells match the queried HLA loci and the queried ABO blood type.

19. The method of any one of claims 11 to 18, wherein the data entry for each of the plurality of characterized cells further comprises RHD blood group and the query further comprises RHD blood group, and wherein the one or more matching data entries of the database representative of the one or more characterized compatible cells match the queried RHD blood group and the queried HLA loci.

20. The method of any one of claims 11-19, wherein the queried HLA loci correspond to the particular subject or particular group of subjects in need of an HLA matched iPSC-derived CAR-T composition.

21. The method of any one of claims 9 to 20, wherein one or more of the queried HLA loci is determined by processing and analyzing a biological sample from the particular subject in need of the HLA match.

22. The method of claim 18, wherein the queried ABO blood type, is determined by processing and analyzing a biological sample from the particular subject in need of an ABO match.

23. The method of claim 19, wherein the queried RHD blood group is determined by processing and analyzing a biological sample from the particular subject in need of a RHD blood group match.

24. The method of any one of claims 10-23, wherein the physical repository comprises one or more liquid nitrogen storage tanks.

25. The method of any one of claims 11-24, comprising producing T cells from each of the one or more characterized compatible cells corresponding to the one or more data entries matching the queried HLA loci.

26. The method of any one of claims 10 to 25, further comprising administering the iPSC-derived CAR-T composition to the particular subject or particular group of subjects.

27. The method of claim 26, wherein the administering step comprises administering the iPSC-derived CAR-T composition to the particular subject or particular group of subjects for treatment of a known disease or condition in the in the particular subject or particular group of subjects.

28. The method of claim 27, wherein the known disease or condition is a cancer.

29. The method of claim 28, wherein the known disease or condition is a cancer comprising a member selected from the group consisting of acute myeloid leukemia, acute lymphoblastic leukemia, chronic myeloid leukemia, chronic lymphocytic leukemia, myeloproliferative disorders, myelodysplastic syndromes, multiple myeloma, non-Hodgkin lymphoma, Hodgkin disease, aplastic anemia, pure red-cell aplasia, paroxysmal nocturnal hemoglobinuria, Fanconi anemia, thalassemia major, sickle cell anemia, severe combined immunodeficiency (SCID), Wiskott-Aldrich syndrome, hemophagocytic lymphohistiocytosis, inborn errors of metabolism, epidermolysis bullosa, severe congenital neutropenia, Shwachman-Diamond syndrome, Diamond-Blackfan anemia, and leukocyte adhesion deficiency.

30. The method of any one of claims 11-29, wherein the database comprises a data entry corresponding to each of a plurality of iPSC super donor cell lines, wherein the data entry for each super donor cell line comprises a set of characterized HLA loci corresponding to the super donor cell line.

31. The method of claim 30, wherein each of the plurality of iPSC super donor cell lines can be used for treatment of a particular subject or particular group of subjects having matching HLA loci with lower risk of immune rejection by the particular subject or particular group of subjects.

32. The method of claim 30 or claim 31, further comprising determining the set of characterized HLA loci corresponding to each of the plurality of super donor cell lines by processing and analyzing one or more biological samples collected from each of one or more super donor individuals.

33. The method of claim 32, wherein the step of determining the set of characterized HLA loci corresponding to each of the plurality of super donor cell lines comprises identifying a set of at least 3 HLA loci, wherein the at least 3 HLA loci are HLA-A, HLA-B, and HLA-DRB.

34. The method of claim 32, wherein the step of determining the set of characterized HLA loci corresponding to each of the plurality of the super donor cell lines comprises identifying a set of at least 9 HLA loci, wherein the at least 9 HLA loci are HLA-A, HLA-B, HLA-C, HLA-DRB1, HLA-DRB3, HLA-DRB4, HLA-DRB5, HLA-DQB1, and HLA-DPB1.

35. The method of claim 34, wherein the set of characterized HLA loci corresponding to each of the plurality of the super donor cell lines comprises at least 3 HLA loci selected from the group consisting of HLA-A, HLA-B, HLA-C, HLA-DRB1, HLA-DRB3, HLA-DRB4, HLA-DRB5, HLA-DQB1, and HLA-DPB1.

36. The method of any one of claims 30 to 35, wherein the set of characterized HLA loci corresponding to each of the plurality of the super donor cell lines are homozygous for HLA-A, HLA-B, and DRB-1.

37. The method of claim 36, wherein the homozygous set of characterized HLA loci belong to a set of most-common HLA loci for a given population that matches a majority of the given population.

38. The method of any one of claims 30-37, wherein the homozygous set of characterized HLA loci comprise homozygous HLA loci in at least 3 major sites, wherein the major sites are members selected from the group consisting of HLA-A, HLA-B, HLA-C, HLA-DRB1, HLA-DRB3, HLA-DRB4, HLA-DRB5, HLA-DQB1, and HLA-DPB1.

39. The method of any one of claims 30-38, wherein the plurality of iPSC super donor cell lines match at least 70% of the population from which the particular subject originates.

40. The method of any one of claims 30-39, wherein the iPSC-derived CAR-T composition is produced using one of the plurality of iPSC super donor cell lines.

41. The method of any one of claims 30-39, comprising exposing the iPSC super donor cell line used to produce the iPSC-derived CAR-T composition to culture media.

42. The method of claim 41, wherein the iPSC-derived CAR-T composition comprises the plurality of compatible T cell lines or the plurality of iPSC super donor cell lines.

43. A composition of matter comprising an iPSC-derived CAR-T composition comprising one or more desired T cells, wherein the composition is produced by the method of any one of the preceding claims.

44. The composition of claim 43, wherein the iPSC-derived CAR-T composition comprises iPSCs and/or iPSC-derived cells derived from a biological sample of a particular subject.

45. The composition of claim 44, wherein the iPSC-derived CAR-T composition is produced using the iPSCs and/or iPSC-derived cells derived from a biological sample of the subject.

46. The composition of any one of claims 43 to 45, wherein the iPSC-derived CAR-T composition comprises compatible cells from a physical repository, wherein the compatible cells are identified as compatible to the particular subject or a particular group of subjects.

47. The composition of claim 46, wherein the compatible cells are identified as compatible with the particular subject or the particular group of subjects using an identification of cell type indicative of compatibility with the particular subject or particular group of subjects, wherein the identification of cell type indicative of compatibility comprises one or more of (i) to (iii): (i) an HLA match, (ii) an ABO blood type match, and (iii) an RHD blood group match having the same HLA loci, and/or ABO blood type, and/or RHD blood group as the particular subject or the particular group of subjects.

48. A method of storing a iPSC-derived CAR-T composition tailored for treatment of a particular subject or particular group of subjects, said method comprising the steps of:
(a) identifying, by a processor of a computing device, as compatible with the particular subject or particular group of subjects, one or more iPSC-derived CAR-T compositions derived using compatible cells corresponding to the one or more iPSCs and/or one or more iPSC-derived cells identified as compatible with the particular subject or particular group of subjects;
(b) labelling, by a processor of a computing device, the one or more iPSC-derived CAR-T compositions with a label, wherein the label comprises information relating to the one or more iPSCs and/or one or more iPSC-derived cells, and a classification of the one or more iPSCs and/or one or more iPSC-derived cells the iPSC-derived CAR-T composition is derived from; and
(c) storing, by a processor of a computing device, a database comprising a data entry corresponding to each label in a physical repository.

49. The method of claim 48, wherein the classification of the one or more iPSCs and/or iPSC-derived cells comprises one or more of (i) to (iii): (i) HLA loci, (ii) ABO blood type, and (iii) RHD blood group.

50. The method of claim 48 or claim 49, wherein the label is a physical label and/or digital label.

51. The method of any one of claims 48 to 50, wherein the label comprises information relating to one or more of (i) to (iii) as follows:
(i) the one or more iPSCs and/or one or more iPSC-derived cells the iPSC-derived CAR-T composition is derived from;
(ii) one or more HLA loci, and/or ABO blood type, and/or RHD blood group compatible with the labeled iPSC-derived CAR-T composition; and
(iii) one or more iPSC-derived CAR-T compositions stored in the physical repository that are compatible with the particular subject or group of subjects, wherein the HLA loci, and/or ABO blood type, and/or RHD blood group of the one or more iPSC-derived CAR-T compositions are identical to or match the HLA loci, and/or ABO blood type, and/or RHD blood group of the one or more iPSCs and/or one or more iPSC-derived cells.

52. A method of retrieving one or more produced, labeled and stored iPSC-derived CAR-T compositions derived using iPSCs and/or iPSC-derived cells, said method comprising the steps of:
(a) identifying, by a processor of a computing device, as compatible with a particular subject or particular group of subjects, one or more iPSC-derived CAR-T compositions derived using one or more iPSCs and/or iPSC-derived cells identified as compatible with the particular subject or particular group of subjects;
(b) retrieving, from a physical repository, the one or more compatible iPSC-derived CAR-T compositions corresponding to the one or more iPSCs and/or one or more iPSC-derived cells identified as compatible with the particular subject or particular group of subjects; and
(c) updating, by a processor of a computing device, a database comprising data entries corresponding to the particular subject or particular group of subjects.

53. The method of claim 52, wherein the retrieved iPSC-derived CAR-T compositions is administered as treatment to the particular subject or particular group of subjects.

54. A method of administering an iPSC-derived CAR-T composition tailored for treatment of a particular subject or particular group of subjects, said method comprising the steps of:
(a) identifying, by a processor of a computing device, the particular subject or particular group of subjects, as having low lymphocyte numbers or poor lymphocyte quality;
(b) identifying, by the processor, as compatible with the particular subject or particular group of subjects, one or more iPSCs and/or one or more iPSC-derived cells;
(c) retrieving compatible cells corresponding to the one or more iPSCs and/or one or more iPSC-derived cells identified as compatible with the particular subject or particular group of subjects;
(d) producing the iPSC-derived CAR-T composition using the retrieved compatible cells; and
(e) administering to the particular subject or particular group of subjects the iPSC-derived CAR-T composition.

55. The method of claim 54, wherein step (c) comprises obtaining the compatible cells from a physical repository.

56. The method of claim 54 or 55, wherein step (c) comprises retrieving the compatible cells using a processor-based query from a user, wherein the query comprises an identification of a cell type indicative of compatibility with the particular subject or particular group of subjects.

57. The method of claim 56, wherein the identification of cell type indicative of compatibility with the particular subject or particular group of subjects comprises one or more of (i) to (iii): (i) an HLA match, (ii) an ABO blood type match, and (iii) an RHD blood group match.

58. The method of any one of claims 54-57, wherein step (b) comprises identifying, one or more stored and labeled iPSC-derived CAR-T compositions within the physical repository derived using one or more iPSCs and/or one or more iPSC-derived cells identified as compatible with the particular subject or particular group of subjects.

59. The method of any one of claims 54-58, wherein step (c) comprises retrieving, the one or more identified iPSC-derived CAR-T compositions corresponding to the one or more iPS cells and/or one or more iPSC-derived cells identified as compatible with the particular subject or particular group of subjects.

60. The method of any one of claims 54-59, wherein the compatible cells are engineered using CRISPR/Cas9 technology.

61. The method of claim 60, wherein step (d) comprises producing the iPSC-derived CAR-T composition using the engineered compatible cells.
